(19) United States
(12) Patent Application Publication
(10) Pub. No.: US 2010/0298840 A1
(43) Pub. Date: Nov. 25, 2010

(54) Phimosis Treatment Device and Method

(76) Inventor: Lyman D. Schwartz, Gettysburg, PA (US)

Correspondence Address: HOOKER & HABIB, P.C., 100 CHESTNUT STREET, SUITE 304, HARRISBURG, PA, 17101, US

(21) Appl. No.: 12/470,870

(22) Filed: May 22, 2009

(51) Int. Cl. A61B 17/32

(52) U.S. Cl. 606/118

(57) ABSTRACT

The invention relates to a medical device and method for treating phimosis. The invention allows for effective, long treatment sessions without interrupting the patient's everyday activities.

### FIELD OF THE INVENTION

The invention relates to a medical device and method for treating phimosis.

### BACKGROUND OF THE INVENTION

Phimosis is a condition wherein the male foreskin cannot be fully retracted from the glans or head of the penis. At birth, the inner layer of the foreskin is sealed to the glans of the penis. In normal development, the foreskin remains non-retractable in infancy and early childhood until puberty, whereupon the foreskin retracts to some degree from the glans to allow the glans to emerge through a slit in the foreskin.

Phimosis can cause discomfort or pain during urination or sexual intercourse. It may also obstruct the release of urine, resulting in ineffective urination or a harmful urinary obstruction.

Known treatments for treating phimosis include circumcision, the surgical removal of the foreskin. This treatment is undesirable as the surgery is costly, intrusive and painful.

Other treatment options include pliers-like tools used to stretch and increase the size of the foreskin slit to allow the glans to emerge through the foreskin slit, such as the tool disclosed in Nishiki US Patent Application Publication No. 2004/0098003. The disclosed tool is undesirable as multiple, dedicated sessions are required to complete treatment. The tool is relatively large and inhibits physical activity during treatment sessions.

Thus, there is a need for a medical device for treating phimosis that is inexpensive, non-intrusive and not painful. The device should be light and small to permit treatment while the patient is active.

### SUMMARY OF THE INVENTION

The invention is a medical device for treating phimosis. The device is a spring-like tool having two ends and a spring for applying a constant force to enlarge the foreskin slit.

The device allows an inexpensive, non-intrusive and non-painful method of treating phimosis. The device is small and lightweight, allowing the patient to remain active during treatment sessions.

Other objects and features of the invention will become apparent as the description proceeds, especially when taken in conjunction with the accompanying drawing sheets illustrating the invention.

### DESCRIPTION OF THE DRAWINGS

FIG. 1 is a top view of the device; and

FIG. 2 is a side view of the device taken along line 2-2 of FIG. 1.

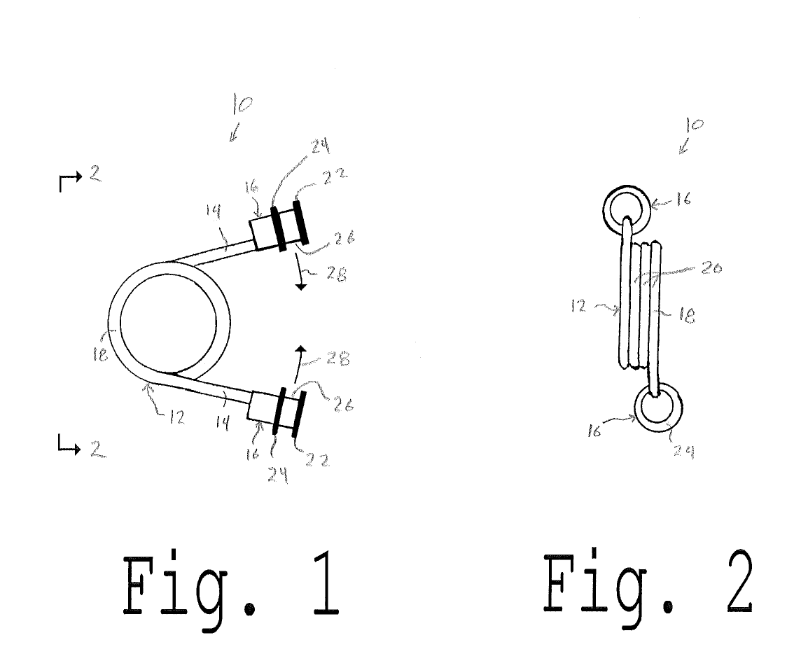

### DETAILED DESCRIPTION OF THE INVENTION

Phimosis treatment device 10 is made up of a compact generally cylindrical body 12 from which two arms 14 extend outwardly from one side of the body to enlarged engaging members 16. Arms 14 may be continuous with and composed of the same material as body 12.

Body 12 is made up of a coiled spring 18 made up of one or more coiled loops 20. Spring 18 may be composed of 1/16 inch diameter wire. The wire may be copper, a copper alloy or other metal, or another resilient material.

Generally cylindrical enlarged engaging members 16 are located at the free ends of arms 14. Each member 16 includes outer and inner cylindrical ribs 22 and 24 that extend outwardly from the member and define an engagement valley 26. Engaging members 16 may be copper, a copper alloy or other metal, or some another malleable substance identical to body 12 and arms 14.

Arms 14 extend outwardly from one side of body 12 to provide a clearance distance between spring 18 and the patient's body. Engaging members 16 are located at a neutral position when spring 18 is uncompressed. As spring 18 is compressed by exerting force on arms 14, each member 16 moves inwardly in the direction of arrows 28 to an application position. When arms 14 are released, the spring moves each member 16 outwardly to return to the neutral position.

In use of device 10 for a treatment session, spring 18 is compressed so that engaging members 16 are brought in proximity to one another in the application position. Members 16 are placed into the foreskin slit and located so that opposing sides of the slit are places into engagement valley 26 on each member. Spring 18 is then released and allowed to decompress so that the members move outwardly to a working position. In the working position, members 16 apply a force onto the opposing ends of the slit to stretch the foreskin to enlarge the foreskin slit. Device 10 is removed from the patient to end the treatment session.

As treatment sessions progress, the skin surrounding the foreskin slit stretches to increase foreskin slit size until the phimosis is cured.

Device 10 is compact and is light, weighing no more than 4 ounces. This allows a patient to apply the device to the body for treatment sessions while simultaneously undertaking normal everyday tasks that would otherwise be impossible with use of larger and heavier phimosis treatment devices. Long treatment sessions may be implemented without the need to keep the patient inactive. Treatment sessions can be implemented discreetly without interruption of normal patient activities.

If desired, before starting a treatment session, one or both arms 14 may be bent to further move engaging members 16 inward or outward to conform the spacing of the arms to foreskin slits of varying sizes.

If desired, a plurality of devices 10 may be used for progressive treatment sessions, each device having a spring 18 having a different spring constant in order to exert gradually increasing spring forces to the foreskin slit. Different spring forces may be achieved though use of springs having different numbers of coiled loops 20.

Device 10 includes a coil spring 18 that biases arms 14 and 16 apart. The arms are extensions of the ends of the coil spring. Other types of springs are contemplated. The engagement members could be pivoted together and forced apart by a longitudinally compressed coil spring. The engaging members could be mounted on the end of a U-shaped spring similar to the spring of a sugar tongs. The engaging members could be attached to the end of a small compressed hydraulic cylinder with a piston bias toward an extended position by a compressed gas. The engaging members could be biased apart by a compressed resilient elastomer spring.

While I have illustrated and described preferred embodiments of my invention, it is understood that there are capable of modification, and I therefore do not wish to be limited to the precise details set forth, but desire to avail myself of such changes and alterations as fall within purview of the following claims.

I claim:

1. A device for treating phimosis, the device comprising a body; two arms extending from the body; each arm including an engaging member distal from the body, the device further comprising a neutral position wherein the engaging members are away from each other, an application position wherein the engaging members are in proximity to each other and a working position located between the neutral position and the application position, the body comprising a spring for biasing the engaging members from the application position or working position to the neutral position, each engaging member including a member for engaging skin on either side of a foreskin slit.

2. The device of claim 1 wherein the spring is comprised of one or more coils.

3. The device of claim 2 wherein the coiled loop is comprised of wire.

4. The device of claim 3 wherein the wire is comprised of metal.

5. The device of claim 4 wherein the metal is copper.

6. The device of claim 5 wherein the wire is generally cylindrical and has a diameter of approximately 1/16 of an inch.

7. The device of claim 3 wherein the arms are wire and are continuous with the body and the spring.

8. The device of claim 3 wherein the means for engaging the skin on either side of a foreskin slit comprises an engagement valley.

9. The device of claim 8 wherein the engagement valley is defined by a pair of ribs, the ribs extending outwardly from the engaging members.

10. The device of claim 10 wherein the device weighs 4 ounces or less.

11. A method for treating a patient's phimosis, the method comprising the steps of:
(a) providing a pair of engagement members, the engagement members biased to a neutral position by a spring force;
(b) compressing a pair of engagement members from the neutral position to an application position;
(c) placing the pair of engagement members within a foreskin slit;
(d) releasing the pair of engagement members within the foreskin slit;
(e) engaging the skin of the foreskin slit with the pair of engagement members;
(f) biasing the pair of engagement members away from each other from the application position to a working position;
(g) exerting a constant force to the skin at the foreskin slit; and
(h) stretching the skin at the foreskin slit to enlarge the foreskin slit.

12. The method of claim 11 further comprising the step of:
(i) allowing the patient to continue everyday tasks simultaneous to the stretching.

13. The method of claim 11 further comprising the steps of:
(i) removing the pair of engagement members from the foreskin slit;
(j) providing a second pair of engagement members, the second pair of engagement members biased to a neutral position by a second spring force greater than the first spring force;
(k) compressing the second pair of engagement members from the neutral position to an application position;
(l) placing the second pair of engagement members within the foreskin slit;
(m) releasing the second pair of engagement members within the foreskin slit;
(n) engaging the skin of the foreskin slit with the second pair of engagement members;
(o) biasing the second pair of engagement members away from each other from the application position to a working position;
(p) exerting a second constant force to the skin at the foreskin slit, the second constant force greater than the first constant force; and
(q) stretching the skin at the foreskin slit to further enlarge the foreskin slit.

14. The method of claim 13 further comprising the step of:
(r) allowing the patient to continue everyday tasks simultaneous to the stretching.
